(19) United States
(12) Patent Application Publication
(10) Pub. No.: US 2023/0233615 A1
(43) Pub. Date: Jul. 27, 2023

(54) PHARMACEUTICAL COMPOSITION FOR TREATING ALZHEIMER'S DISEASE

(71) Applicants: Gwo Xi Stem Cell Applied Technology Co., Ltd., Zhubei (TW); National Yang Ming Chiao Tung University, Hsinchu (TW)

(72) Inventors: Chi-Ying Huang, Zhubei (TW); Yu-Chen Tsai, Zhubei (TW); Ming-Hsi Chuang, Zhubei (TW); Po-Cheng Lin, Zhubei (TW)

(21) Appl. No.: 18/097,509

(22) Filed: Jan. 17, 2023

Related U.S. Application Data
(60) Provisional application No. 63/303,494, filed on Jan. 26, 2022.

(51) Int. Cl. A61K 35/28; A61P 25/28; C12N 15/113; C12N 9/64; C12N 5/0775

(52) U.S. Cl. CPC A61K 35/28; A61P 25/28; C12N 5/0667; C12N 9/6494; C12N 15/113; C12Y 304/24011; C12N 2310/141; C12N 2310/531; C12N 2500/32; C12N 2500/42

(57) ABSTRACT

The present disclosure provides a pharmaceutical composition for treating Alzheimer’s disease, which at least includes an extracellular vesicle that is prepared by a method including: a first culturing step: performing an amplification culture of an adipose-derived stem cell in a first culture medium at a cell density of 6,000-15,000 cells/cm² until an amplification amount of the adipose-derived stem cell is above 90% of that before the culture; a second culturing step: culturing the amplified adipose-derived stem cell in a second culture medium at a cell density of 10,000-100,000 cells/cm² for 20-30 hours; and an extracellular vesicle separating step: collecting a culture solution and separating the extracellular vesicle from the culture solution by utilizing a tangential flow filtration (TFF) or ultrafiltration method.

### CROSS-REFERENCE TO RELATED APPLICATION

The present application claims priority from U.S. Provisional Pat. Application No. 63/303,494 filed on Jan. 26, 2022, the contents of which are incorporated herein by reference in their entirety.

### FIELD OF TECHNOLOGY

The present disclosure relates to a pharmaceutical composition for treating Alzheimer’s disease, in particular to a pharmaceutical composition of a high-phenotype mesenchymal stem cell and/or an extracellular vesicle derived from the high-phenotype mesenchymal stem cell capable of highly expressing a neprilysin, miR-29a and/or miR-29b.

### BACKGROUND

Amyloidosis is an acquired or inherited disease and caused by abnormal deposition of beta-sheet fibrillar protein aggregates in various tissues. Amyloidosis is classified into primary amyloidosis (AL), secondary amyloidosis (AA), and hereditary amyloidosis (ATTR). This disease can be local or systemic. Amyloid accumulates in different organs such as nerves, liver, kidneys, spleen and blood vessels, causing different clinical symptoms, including familial amyloid polyneuropathy, tenosynovitis, familial amyloidosis, cranial nerve disease, hereditary cerebral hemorrhage, hereditary spongiform encephalopathy, renal disease (chronic dialysis disease), deafness, urticaria, limb pains, cardiomyopathy, skin serration, multiple myeloma, macroglobulinemia, myelomatosis dependency amyloidosis, Tiroidina bone marrow cancer, early/non-genetic/genetic Alzheimer’s disease (AD), Parkinson’s disease, Huntington’s disease, Creutzfeldt-Jakob disease, cerebral amyloid angiopathy, inclusion body myositis, macular degeneration, mild cognitive damage or mild or moderate cognitive impairment, the cognitive degradation related with the age, Down’s syndrome, diabetes, vascular dementia, senile dementia, AA amyloidosis, AL amyloidosis, hemodialysis-associated amyloidosis or hereditary cerebral hemorrhage, etc.

Alzheimer’s disease (AD) is one of the most common neurodegenerative diseases, and leads to a series of phenotypes, including memory deterioration and other cognitive impairments, and ultimately even death. About 50 million people worldwide are affected by dementia in 2020 and 1,350 million people are expected to be affected in 2050. Main pathological features of Alzheimer’s disease are extracellular amyloid (beta amyloid, Aβ) plaque aggregation and hyperphosphorylation of an intracellular Tau protein to form neurofibrillary tangles (NFTs), which results in synaptic loss, communication outage of cortex and hippocampus, and neuronal death, thereby affecting impaired memory and learning abilities. There is currently no effective treatment means or drug that can inhibit amyloid deposition (Aβ deposition), neuronal death and memory deterioration and cognitive impairment.

Neuroprotection is currently performed by regulating a microglia function by reducing neuroinflammation and oxidative stress or giving growth factors. However, these strategies fail to exert both neuroprotective and neurogenesis-inductive efficacies. In addition, a blood-brain barrier (BBB) will prevent a neuroprotective agent from entering a central nervous system (CNS). In recent years, it is found that mesenchymal stem cells (MSCs) have been a new choice for treating central nervous system diseases. MSCs can home to a damaged area and can differentiate into functional cells to directly replace damaged cells. Meanwhile, MSCs have characteristics of easy isolation and expansion, self-renew, multilineage differentiation, low immunogenicity, and immunoregulation. In addition, transplantation of MSCs has been shown to alleviate a disease progression of amyotrophic lateral sclerosis. Therefore, it is concluded that MSCs have a potential for treating neurodegenerative diseases.

However, use of MSCs is still largely limited by their rapid clearance from the circulation and the harsh microenvironment at an injury site preventing their survival. It is further found that MSCs transplantation can only exert a short-term effect. In addition, side effects and safety of MSCs are still controversial. At present, a research has shown that MSCs exert their repair effect mainly through a paracrine effect. MSCs secrete various bioactive molecules such as growth factors, proteins, cytokine signaling lipids, mRNA, and small ribonucleic acids (microRNA, miRNA), which are carried to target cells via extracellular vesicles (EVs). EVs are an important mediator of an intercellular communication and involved in regulating physiological processes and occurrence of diseases. In comparison to MSCs, MSCs-derived EVs do not have functional nuclei, cannot replicate, and divide uncontrollably, thereby reducing a risk of genetic disruption during their isolation and expansion. At the same time, MSCs-derived EVs fill a gap of a cell-free therapy by providing higher safety and lower immunogenicity and have similar effects on immune regulation and regeneration. Therefore, MSCs-derived EVs would be an alternative to cell transplantation for disease treatment.

MSCs-derived EVs are a heterogeneous particle, are formed by outward budding or exocytosis of primitive cells, include exosomes and microvesicles (MVs), and are nano-sized particles with phospholipid bilayers secreted by cells. EVs have a diameter range of 30 nm to 1 µm. EVs are rich in tetraspanin proteins on a cell surface, mainly CD9, CD63, CD81 and other proteins, such as an ALG-2 interacting protein-X (Alix) and a tumor susceptibility gene 101 (TSG 101). Alix, also known as a programmed cell death 6 interacting protein (PDCD6IP), is an adaptor protein that binds to ESCRT and endophilin-A proteins. Alix is expressed in neurons and concentrates at synapses during epileptic seizures. TSG101 and signal-transducing adaptor molecules play a key role in exosome biogenesis and secretion. Researches have shown that large-scale expression of TSG101 in neural stem cells can increase related genes for exosome biogenesis, thereby improving exosome secretion. EVs can facilitate intercellular communication, including BBB. Researches have shown that in an Alzheimer’s disease model, specific cells preferentially release EVs, wherein some EVs provide a certain degree of neuroprotection.

However, a clinical development of extracellular vesicles as a therapeutic drug or a drug delivery platform requires production of a large amount of extracellular vesicles. Besides, how to encapsulate a drug or a gene that can treat a disease into the extracellular vesicles remains a challenge. To date, this situation has hampered researches on assessing a preclinical efficacy. In general, exosome production in support of a complete animal model study may take several months. A gold standard for an isolation of exosomes usually requires four to five consecutive high-speed or ultra centrifugation steps. The exosomes obtained by the ultracentrifugation purification are on average 500-1,000 particles per cell. But these methods are not extendable and have a low recovery rate.

### SUMMARY

In view of this, the present disclosure aims to provide a pharmaceutical composition for treating Alzheimer’s disease. An extracellular vesicle derived from an adipose-derived stem cell is cultured in a culture medium free of an animal serum or a xenogenic animal material or any xenogenic animal-derived derivative in a xeno-free cell culture environment. Besides, a large amount of extracellular vesicles can be stably obtained. For example, in a specific example, a yield of the extracellular vesicles is 500-2,500, which is 2-5 times that of a conventional process. An mRNA of a neprilysin, miR-29a and/or miR-29b can further be transfected into the cell using nano-channel electroporation (NEP) during the culture process to improve a therapeutic effect.

Specifically, the present disclosure provides a pharmaceutical composition for treating Alzheimer’s disease, which at least includes an extracellular vesicle that is prepared by a method including: a first culturing step: performing an amplification culture of an adipose-derived stem cell in a first culture medium at a cell density of 6,000-15,000 cells/cm² until an amplification amount of the adipose-derived stem cell is above 90% of that before the culture; a second culturing step: culturing the amplified adipose-derived stem cell in a second culture medium at a cell density of 10,000-100,000 cells/cm² for 20-30 hours; and an extracellular vesicle separating step: collecting a culture solution and separating the extracellular vesicle from the culture solution by utilizing a tangential flow filtration (TFF) or ultrafiltration method, wherein the first culture medium is a culture medium containing fetal bovine serum, N-acetyl-L-cysteine, L-ascorbic acid 2-phosphate, and Keratinocyte-SFM; and the second culture medium is a culture medium containing N-acetyl-L-cysteine, L-ascorbic acid 2-phosphate, and Keratinocyte-SFM.

According to an example of the present disclosure, after the second culturing step, the method further comprises: a gene transfection step: spreading the adipose-derived stem cell on a surface of a 3D NEP silicon chip, after the cell is adhered, treating the cell by using an electroporation system under a condition of a square-wave pulsed electric field of 120-150 V and a duration of 10 ms for 5-15 times of pulses, injecting a plasmid added in a PBS buffer solution in advance into a single cell through a nano-channel, and culturing the cell for 20-30 hours to perform the extracellular vesicle separating step.

According to an example of the present disclosure, the plasmid in the PBS has a concentration between 400-600 ng/ml.

According to an example of the present disclosure, the plasmid is an mRNA of a neprilysin, miR-29a and/or miR-29b

According to an example of the present disclosure, the extracellular vesicle highly expresses a neprilysin, miR-29a and/or miR-29b.

According to an example of the present disclosure, the pharmaceutical composition is administered transnasally.

In addition, the present disclosure can further provide a method for culturing a high-phenotype mesenchymal stem cell. The method comprises the following steps: a first culturing step: performing an amplification culture of an adipose-derived stem cell in a first culture medium at a cell density of 6,000-15,000 cells/cm² until an amplification amount of the adipose-derived stem cell is above 90% of that before the culture; a second culturing step: culturing the amplified adipose-derived stem cell in a second culture medium at a cell density of 10,000-100,000 cells/cm² for 20-30 hours; and a gene transfection step: spreading the adipose-derived stem cell on a surface of a 3D NEP silicon chip, after the cell is adhered, treating the cell by using an electroporation system under a condition of a square-wave pulsed electric field of 120-150 V and a duration of 10 ms for 5-15 times of pulses, injecting a plasmid added in a PBS buffer solution in advance into a single cell through a nano-channel, and culturing the cell for 20-30 hours to obtain a high-phenotype mesenchymal stem cell, wherein the plasmid is an mRNA of a neprilysin, miR-29a and/or miR-29b; and the high-phenotype mesenchymal stem cell highly expresses the neprilysin, miR-29a and/or miR-29b.

According to an example of the present disclosure, the high-phenotype mesenchymal stem cell and/or the extracellular vesicle derived from the high-phenotype mesenchymal stem cell can be used for treating Alzheimer’s disease.

### BRIEF DESCRIPTION OF THE DRAWINGS

FIG. 1A shows a particle size analysis of EVs derived from ADSCs in example 1;

FIGS. 1B and 1C show results of a western blotting analysis in example 1 respectively;

FIGS. 2A and 2B show comparisons of levels of NO and TNF-α in an analysis of an influence of EVs on neuroinflammation respectively;

FIGS. 3A to 3E show an operation process of an intranasal route (IN route) administration analysis, distribution of PKH26-labeled control group (PBS) or EV in body of mice, distribution of PKH26-labeled control group (PBS) or EV in organs of mice, comparison of the fluorescence amount in the mice, and comparison of the fluorescence amount of mouse organs respectively;

FIGS. 4A and 4B show an operation time course of operation and changes in body weight of mice in a side effect analysis respectively;

FIGS. 5A to 5F show mouse nest building photos and a scoring schematic diagram in an animal behavior test analysis, comparisons of scores and nest weights of each group in a nest building test analysis, and comparisons of burrowed food weight in 2 hours and 24 hours of each group in a burrowing test respectively;

FIGS. 6A to 6C show schematic diagrams, comparisons of total exploration time and a discrimination index in an animal memory test analysis respectively;

FIGS. 7A and 7B show a local enlargement of cortex regions in an Aβ plaque loading analysis of APP/PS1 mice and a quantitative comparison of an Aβ plaque relative intensity of whole hemisphere respectively;

FIG. 8 shows immunohistochemical staining results in an analysis of astrocyte proliferation and microglia proliferation of APP/PS1 mice respectively, wherein A in FIG. 8 shows detection of activated astrocytes, GFAP (blue) and DE2 (Aβ1-16, green) in a cortex of WT+PBS, WT+ADSC-EV, APP/PS1+PBS (AD+PBS), and APP/PS1+ADSC-EV (AD+ADSC-EV) by an immunohistochemical staining; and B in FIG. 8 is a GFAP expression analysis of a cortical lysate by a western blotting;

FIGS. 9A and 9B show immunohistochemical staining results in an analysis of astrocyte proliferation and microglia proliferation of APP/PS1 mice, wherein FIG. 9A shows activated microglia in a hippocampus of WT+PBS, WT+ADSC-EV, APP/PS1+PBS (AD+PBS), and APP/PS1+ADSC-EV (AD+ADSC-EV) through detection of Iba-1 (red) and DE2 (Aβ1-16, green) by an immunohistochemical staining, and data are mean±SEM; and FIG. 9B shows quantitative data results of FIG. 9A; and

FIGS. 10A to 10C show a partially enlarged view of the SGZ in dentate gyrus, comparisons of the number of BrdU-positive cells, and the number of DCX-positive cells respectively in an analysis of neurogenesis by a transnasal administration in vivo.

### DESCRIPTION OF THE EMBODIMENTS

In order to enable the objective, technical features and advantages of the present disclosure to be more understood by a person skilled in the art to implement the present disclosure, the present disclosure is further illustrated by accompanying the appended drawings, specifically clarifying technical features and embodiments of the present disclosure, and enumerating better examples. In order to express the meaning related to the features of the present disclosure, the corresponding drawings herein below are not and do not need to be completely drawn according to the actual situation.

All technical and scientific terms used herein have the same meanings as those generally understood by a person of ordinary skill in the art to which the present disclosure pertains. Furthermore, as used herein, singular terms shall include a plural form and plural terms shall include a singular form, unless otherwise clearly contradicted by context.

Although the numerical ranges and parameters defining a broader scope of the present disclosure are all approximations. The related numerical values in the specific examples are presented herein as precisely as possible. However, any numerical value essentially inevitably contains standard deviations caused by an individual measuring method. As used herein, the term “about” generally refers to an actual value within plus or minus 10%, 5%, 1% or 0.5% of a specific value or range. Alternatively, the term “about” indicates that an actual value falls within an acceptable standard deviation of an average value, subjecting to consideration by a person with common knowledge in the art to which the present disclosure pertains. In addition to the examples or unless otherwise specified, all the ranges, amounts, values and percentages (e.g., to describe amounts of materials, length of time, temperatures, operating conditions, quantitative proportions, and the like) herein used are to be understood as modified by the term “about”. Accordingly, unless indicated to the contrary, the numerical parameters set forth in the description and attached claims are approximations and may be changed as required. At least, these numerical parameters are to be understood as values obtained by the indicated number of significant digits and a general carry method.

In order to make the description of the present disclosure more detailed and complete, the following provides an illustrative description for the embodiments and specific examples of the present disclosure; but this is not the only form of implementing or using the specific examples of the present disclosure. The embodiments cover the features of the various specific examples as well as method steps and sequences for constructing and operating these specific examples. However, other examples may further be used to achieve the same or equivalent functions and step sequences.

Then, the present disclosure is described below with reference to the specific examples and comparative examples.

### Example 1

In this example, adipose-derived stem cells (ADSCs) and Wharton’s Jelly mesenchymal stem cells (WJ-MSCs) were used to obtain extracellular vesicles respectively.

The adipose-derived stem cells or Wharton’s Jelly mesenchymal stem cells at 6,000-15,000 cells/cm² were cultured in a Keratinocyte-SFM medium containing 10 wt% of fetal bovine serum, 1-100 mM of N-acetyl-L-cysteine, and 0.05-50 mM of L-ascorbyl acid 2-phosphate for 5-14 days to expand the number of the cells to 10,000-100,000, wherein the culture was performed at a temperature controlled at 36.5-38.5° C. and in a cell incubator containing 5% carbon dioxide.

Then the cells were rinsed twice with Dulbecco’s phosphate buffered saline (DPBS), then the stem cells were cultured in a Keratinocyte-SFM culture medium containing 1-100 mM of N-acetyl-L-cysteine and 0.05-50 mM of L-ascorbyl acid 2-phosphate for 24 hours at a temperature controlled at 36.5-38.5° C. and in a cell incubator containing 5% carbon dioxide.

A culture solution after the completion of the culture was collected and centrifuged at a centrifugation speed of 300 g at a temperature of 4° C. for 5 min to remove dead cells in the culture solution; and then after a supernatant was collected, the supernatant was centrifuged at a centrifugation speed of 2,000 g at a temperature of 4° C. for 20 min to remove cell debris, then the supernatant was collected and filtered through a filter screen having a pore size of 0.22 µm, extracellular vesicles were separated from the filtered culture solution by tangential flow filtration (TFF) or ultrafiltration (Amicon UF) using different molecular weight cut off (MWCO), and a size distribution and a concentration of the extracellular vesicles were confirmed by a nanoparticule tracking analysis.

### Effects of Extracellular Vesicles on Human Neuroblastoma SH-SY5Y APP695 Cells

Amyloid plaque is one of marks of pathology of Alzheimer’s disease. Aβ is prepared by cutting and hydrolyzing amyloid precursor protein (APP) through an enzyme. APP produces a soluble APP (sAPPα) and CTFα released from a cell surface by α-secretase. β-secretase can cleave APP, generating sAPPβ and CTFβ. A γ-secretase can directly or indirectly produce Aβ peptides of various sequence lengths, i.e., Aβ40 and AP42, wherein Aβ42 is more hydrophobic and more prone to aggregation, produces neurotoxicity, and leads to neurodegeneration and neuronal death. An interaction between Aβ42 and Aβ40 is generally considered to play a key role in Alzheimer’s disease. An increased Aβ42/Aβ40 ratio appears to correlate with cases of early-onset familial Alzheimer’s disease caused by presenilin mutations. Decreasing the Aβ42/Aβ40 ratio in transgenic mice decreases Aβ deposition. Studies have shown that a higher Aβ42/Aβ40 ratio is more neurotoxic.

An SH-SY5YAPP695 cell line used in this example is as shown in “Yuan-Hu Zhi Tong Prescription Mitigates Tau Pathology and Alleviates Memory Deficiency in the Preclinical Models of Alzheimer’s Disease”; Front Pharmacol. 2020 Oct 30; 11:584770. Human neuroblastoma SH-SY5Y APP695 cells overexpress APP695 and may produce a high level of Aβ and thus can be used as a drug screening platform for Aβ accumulation. In this example, SH-SY5Y APP695 cells were grown in a DMEM/F12 (GIBCO) culture medium supplemented with 10% exosome-depleted fetal bovine serum, penicillin (100 IU/ml), and streptomycin (10 µg/ml) at 37° C., 5% carbon dioxide, and an appropriate humidity, and screened by a G418 antibiotic. Then SH-SY5Y APP695 cells were inoculated in a 24-well plate for 24 hours, the culture medium was replaced with a fresh culture medium, and exosomes were added in amounts and sources shown in Table 1 for 48 hours of culture. Finally, cell activities of each group were analyzed and supernatants were collected to confirm expression degrees of Aβ1-40 and Aβ1-42 (analyzed by an ELISA assay). The results were recorded in Table 1.

TABLE 1

| Groups | Amount of EVs | Separation method (Method and MWCO) | Sources of EVs | Cell viability (%) | Expression degree (%) of Aβ1-40 | Expression degree (%) of Aβ1-42 | Ratio of Aβ1-2/Aβ1-40 |
|---|---|---|---|---|---|---|---|
| 1 | 4.56E+08 | Amicon UF, 100 kDa | ADSCs#1 | 146.1 | 50.4 | 68.5 | 1.359 |
| 2 | 5.00E+08 | TFF, 500 kDa | ADSCs#1 | 104.8 | 50.6 | 73.7 | 1.457 |
| 3 | 1.00E+09 | TFF, 500 kDa | ADSCs#1 | 132.2 | 72 | 49.1 | 0.682 |
| 4 | 1.00E+09 | Amicon UF, 3 kDa | WJ-MSCs | 180.9 | 131.9 | 200.6 | 1.521 |
| 5 | 3.33E+09 | Amicon UF, 3 kDa | WJ-MSCs | 167.0 | 142.5 | 205.2 | 1.440 |
| 6 | 1.00E+10 | Amicon UF, 3 kDa | WJ-MSCs | 172.2 | 139.3 | 194.7 | 1.398 |
| 7 | 1.00E+09 | Amicon UF, 3 kDa | WJ-MSCs | 172.2 | 137.8 | 228.9 | 1.661 |
| 8 | 1.00E+09 | Amicon UF, 100 kDa | WJ-MSCs | 174.8 | 116.2 | 110.8 | 0.954 |
| 9 | 3.33E+09 | Amicon UF, 100 kDa | WJ-MSCs | 181.7 | 126.4 | 139.0 | 1.100 |
| 10 | 5.00E+08 | TFF, 100 kDa | WJ-MSCs | 141.7 | 96.4 | 84.8 | 0.880 |
| 11 | 1.00E+09 | TFF, 100 kDa | WJ-MSCs | 147.0 | 92.5 | 84.3 | 0.911 |

SH-SY5Y-APP695 cells were administered with different EVs. It was found that a cell viability of the cells administrated with a high dose of 10⁹ particle ADSCs-EVs (No. 3) was increased to 132.2% and higher than 104.3% of the cells administrated with a low dose of 5×10⁸ particle ADSCs-EVs (No. 2). In addition, since Amicon UF has a lower permeability, EVs separated and purified by Amicon UF have a higher cell viability (No.1 v.s. Nos. 2-3; and No.1 v.s. Nos. 4-9 and Nos. 10-11) than EVs separated and purified by TFF. Therefore, the Amicon UF can maintain a high concentration of secreted proteins of MSCs. Secretomes of MSCs contain various growth factors, such as endothelial growth factor (EGF), fibroblast growth factor-2 (FGF-2), etc., which play roles in cell proliferation and survival. Thus, the increase in the cell activity of the EVs separated and purified by the Amicon UF was caused by EVs and other secretomes.

Furthermore, as shown in Table 1, in the ADSC-derived EVs groups (Nos. 1-3) and the WJ-MSCs-derived EVs groups (Nos. 4-11), extracellular Aβ1-40 decreased to 50.4%, 50.6%, 72.0%, 131.9%, 142.5%, 139.3%, 137.8%, 118.2%, 126.4%, 96.4% and 92.5% respectively; and in the ADSC-derived EVs groups (Nos. 1-3) and the WJ-MSCs-derived EVs groups (Nos. 4-11), extracellular Aβ1-42 decreased to 68.5%, 73.7%, 49.1%, 200.6%, 205.2%, 194.7%, 228.9%, 110.8%, 139.0%, 84.8% and 84.3%, respectively. The results showed that the ADSC-derived EVs had better effects on reducing extracellular Aβ1-40 and Aβ1-42 than the WJ-MSCs-derived EVs. Besides, compared with the Amicon UF separation and purification method, the TFF separation and purification method has better effects on reducing extracellular Aβ1-40 and Aβ1-42 in the ADSC-derived EVs groups and the WJ-MSCs-derived EVs groups. As found in Table 1 when comparing Nos. 4-7 (3 kDa) and Nos. 8-9 (100 kDa), EVs separated by a membrane with a lower molecular weight cut off (MWCO) showed a lower effect of reducing extracellular Aβ1-40 and Aβ1-42. These results showed that reducing Aβ was by EVs rather than secretomes. Therefore, it can be known that EVs for treating Alzheimer’s disease had a better source from ADSCs than WJ-MSCs, the TFF separation and purification mode was a better separation method of EVs, and a membrane with a molecular weight cut off of 500 kDa had a better separation effect than that with 3 kDa and 100 kDa.

Then, EVs derived from different ADSC sources were analyzed and compared to EVs derived from WJ-MSCs. The results were shown in Table 2.

TABLE 2

| Groups | Amount of EVs | Separation method (Method and MWCO) | Sources of EVs | Cell viability (%) | Expression degree (%) of Aβ1-40 | Aβ1-42 Expression degree (%) | Ratio of Aβ1-42/Aβ1-40 |
|---|---|---|---|---|---|---|---|
| 2 | 5.00E+08 | TFF, 500 kDa | ADSCs#1 | 104.8 | 50.6 | 73.7 | 1.457 |
| 3 | 1.00E+09 | TFF, 500 kDa | ADSCs#1 | 132.2 | 72 | 49.1 | 0.682 |
| 12 | 5.00E+08 | TFF, 500 kDa | ADSCs#2 | 100.0 | 90 | 99.2 | 1.102 |
| 13 | 1.00E+09 | TFF, 500 kDa | ADSCs#2 | 110.0 | 60 | 72 | 1.200 |
| 14 | 5.00E+08 | TFF, 500 kDa | WJ-MSCs | 105 | 86.8 | 81.4 | 0.938 |
| 15 | 1.00E+09 | TFF, 500 kDa | WJ-MSCs | 108 | 94.8 | 72.1 | 0.761 |
| 16 | 5.00E+09 | TFF, 500 kDa | WJ-MSCs | 109 | 102.4 | 55.3 | 0.540 |

As can be seen from the results in Table 2, the stem cell-derived EVs tended to increase the cell viability at different cell densities. However, the ADSCs#1-derived EVs had the best effects on clearing Aβ1-40 and Aβ1-42, and the effects were more significant as a dose of the ADSCs#1-derived EVs increased.

ADSCs#1 were from individuals at 20-70 years old and at the same time do not have a risk of relevant communicable disease agents or diseases (RCSADs), nor a risk of disease transmission by xenotransplantation. Test results of related infectious pathogens or diseases, such as HIV type 1&2, hepatitis B virus (HBV), hepatitis C virus (HCV), treponema pallidum, human transmissible spongiform encephalopathy (TSE), were negative or non-reactive.

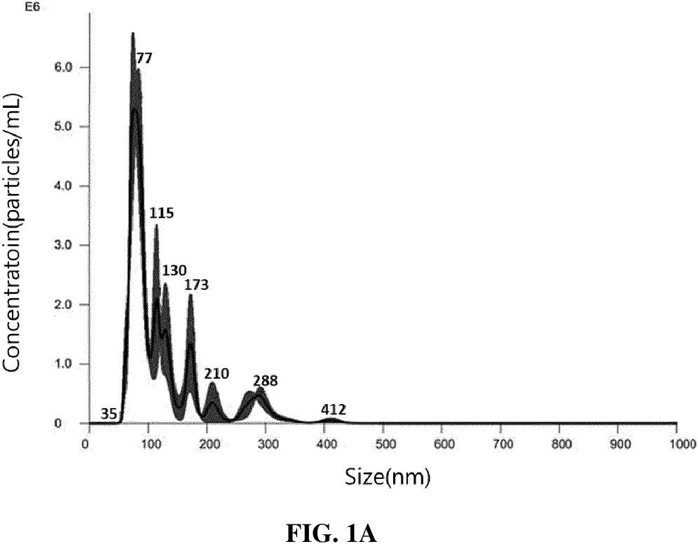

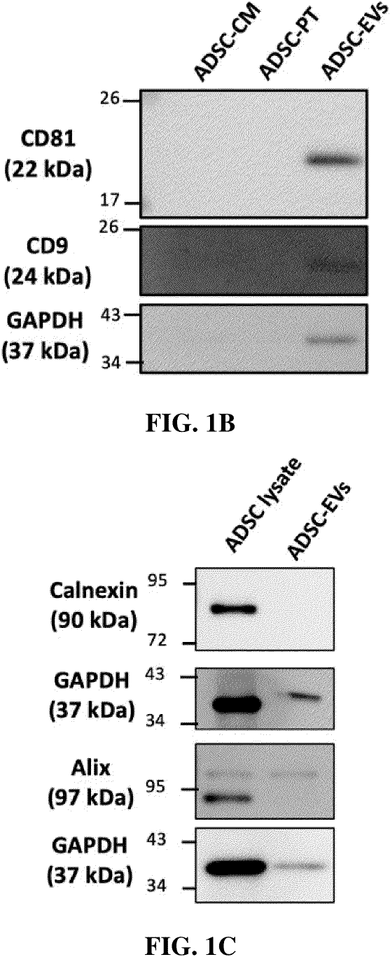

A nanoparticle tracking analysis showed EVs derived from ADSCs had an average particle size of 124.1±2.8 nm, a mode size of 78.4±3.2 nm, and a distribution range of 30 nm to 300 nm, as shown in FIG. 1A. In addition, according to results of western blotting, EVs derived from ADSCs had CD81 and CD9 markers which were not found in a culture medium before the start of the culture nor a liquid after separation and purification by TFF. As shown in FIG. 1B, ADSC-CM indicated the culture medium before the start of the culture, ADSC-PT indicated the liquid after separation and purification by TFF, and ADSC-EVs indicated extracellular vesicles obtained after separation and purification.

In addition, when compared with an ADSC lysate, it can be seen that extracellular vesicles derived from ADSCs (ADSC-EVs) only expressed Alix (a positive marker of extracellular vesicles), but did not express Calnexin (a negative marker of extracellular vesicles) as shown in FIG. 1C. From the above results, it can be known that the method of the present disclosure can successfully separate extracellular vesicles from ADSCs.

In addition, the extracellular vesicles derived from ADSCs (ADSC-EVs) also contain miRNA with a neuroprotective activity, wherein miR-29 reduces production of Aβ, miR-133b and miR-17-92 cluster enhance neurite growth or neurogenesis, and miR-125b and miR-21-5p promote cell survival. In addition, a number of proteins are found to have neuroprotective effects in extracellular vesicles, including neprilysin, neuroplastin and eIF5A. Neprilysin (also called membrane metalloendopeptidase, MME) is one of Aβ-degrading enzymes mainly used for degrading Aβ.

### Analysis of Effects of EVs on Neuroinflammation

Primary mixed glial cells were isolated from the cortex of 3-day-old C57BL/6 mice and contacted with lipopolysaccharide (LPS) (100 ng/ml) for 30 min to trigger neuroinflammation. Then the untreated and LPS-stimulated primary mixed glia cells were co-cultured with extracellular vesicles derived from ADSCs, extracellular vesicles derived from WJ-MSCs, or dexamethasone (10 ng/ml) for 24 hours. The content of TNFα and NO was measured by an enzyme-linked immunosorbent assay (ELISA).

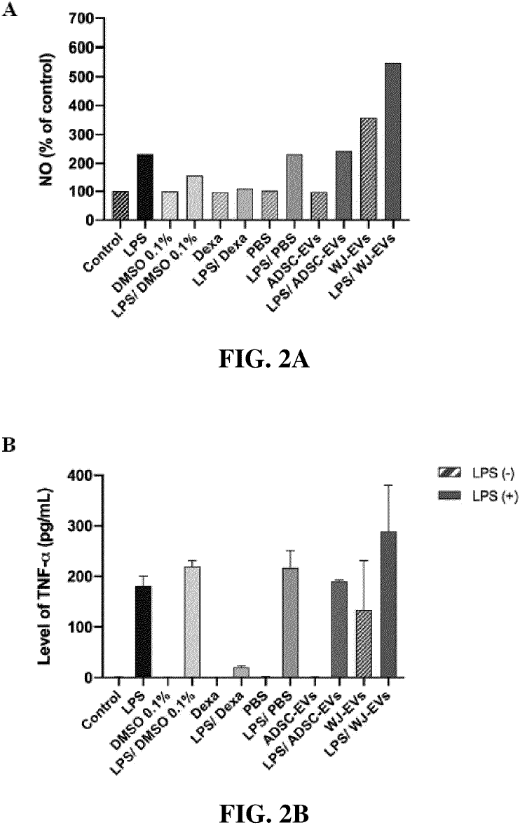

Nitric oxide (NO) and tumor necrosis factor α (TNF-α) act like a double-edged sword in neuroinflammation. Normal levels of NO and TNF-α contribute positively to an increasing number of biological pathways. Excessive NO and TNF-α production is thought to be a main cause of pathogenesis of several neurodegenerative diseases. As a well-known anti-inflammatory agent, dexamethasone reduced NO and TNF- α levels only in inflammatory cases compared to treatment with 0.1% of DMSO. Treatment of ADSC-derived EVs maintained the same levels of NO and TNF-α as a PBS control group after triggering neuroinflammation by LPS. In contrast, WJ-MSCs-derived EVs increased NO production and released TNF- α from mixed glial cells not only under a neuroinflammatory condition, but also prior to the LPS treatment (as shown in FIGS. 2A and 2B). Alzheimer’s disease is a chronic progression requiring a long-term use of a therapeutic agent. Thus, significant overproduction of NO and TNF- α by the WJ-MSCs-derived EVs may be more appropriate for an acute phase of neuroinflammation, but not for a long-term treatment. Treatment of ADSC-derived EVs having unchanged NO and TNF-α levels suggested that the role of the ADSC-derived EVs may be through other neuroinflammatory pathways.

### Analysis of Inhibition on NLRP3 by EVs

In order to investigate anti-inflammatory and NLR family pyrindomain containing 3 (NLRP3) inflammasome inhibitory effects of ADSC-derived EVs, a study was performed using a BV2 microglia cell line (hereinafter referred to as BV2 cells). BV2 cells were initiated with 100 ng/ml of LPS and 5 mM of ATP, and 100 mM of mefenamic acid (a positive control group) or a low dose of ADSC-derived EVs (2×10⁵ particles/cell) or a high dose of ADSC-derived EVs (2×10⁶ particles/cell) in a humidified environment with 5%CO₂ at 37° C. for 48 hours.

Neuroinflammation is also one of causes of Alzheimer’s disease. In the process of neuroinflammation, release of pro-inflammatory cytokines (TNF) is increased, including tumor necrosis factor (TNF), interleukin (IL)-1β, IL-6, IL-18 or chemokine, such as chemokine (C-C motif) and chemokine (C-X-C motif) ligands (CCL1, CCL5, CXCL1, etc.).

In recent years, the research has found that an abnormal expression of an NLRP3 inflammasome is directly related to pathogenesis of Alzheimer’s disease. NLRP3 inflammasome affects Aβ clearance and contributes to the formation of Aβ plaque clusters. NLRP3 inflammasome is a protein complex comprising NLRP3, an apoptosis-associated speck-like protein containing a CARD (ASC) and caspase-1, and plays an important role in an inflammatory response by regulating an activity of caspase-1 and maturation and secretion of inflammatory hormones IL-1β and IL-18. Inflammasomes are composed of a number of proteins, including NOD-like receptors (NLRs), an apoptosis-associated speck-like protein containing a CARD (ASC), and caspase-1. After the inflammasomes are activated, an interleukin 1β precursor (proIL-1β) and an IL-18 precursor (proIL-18) are cleaved into active IL-1β and IL-18.

NLRP3 inflammasome activation must be subjected to priming and activation. Microglia cells are first triggered by binding of PAMP and DAMP to a cell surface TLR4 receptor, and then an NF-κB pathway is activated. It subsequently leads to an upregulation of NLRP3 and to releases of cytokines containing pro-IL-1β and pro-IL-18. Signal 2 triggers the NLRP3 inflammasome by multiple stimulants. Pro-caspase-1 is cleaved into caspase-1 through a pyrin domain interaction and ASC of pro-caspase-1, which in turn cleaves pro-IL-1β and pro-IL-18 into IL-1β and IL-18. The fully active IL-1β and IL-18 are secreted into an extracellular space, thereby triggering neuroinflammation.

The results showed that expressions of IL-1β and IL-18 can be effectively inhibited by ADSC-derived EVs in a priming stage and an activation stage, and the ADSC-derived EVs have a better inhibiting effect on IL-18 than the positive control group (the mefenamic acid group). The mefenamic acid group, 2×10⁵ ADSC-derived EVs group, and 2×10⁶ ADSC-derived EVs group inhibited an expression of IL-1β by 35.4%, 30.5%, and 41.0% respectively (compared to a PBS group). The mefenamic acid group, 2×10⁵ ADSC-derived EVs group, and 2×10⁶ ADSC-derived EVs group inhibited an expression of IL-18 by 29.2%, 36.1%, and 57.9% respectively (compared to the PBS group). The mefenamic acid group, 2×10⁵ ADSC-derived EVs group, and 2×10⁶ ADSC-derived EVs group inhibited an expression of IL-1β by 69.5%, 51.1%, and 78.5% respectively (compared to the PBS group). The 2×10⁵ ADSC-derived EVs group and 2×10⁶ ADSC-derived EVs group inhibited an expression of IL-18 by 32.8% and 68.2% respectively, and the mefenamic acid group had no significant change in the expression of IL-18 (compared to the PBS group).

Roles of the ADSC-derived EVs in the cortex and hippocampus of APP/PS1 mice were further investigated. The mice were divided into 4 groups (n = 3-4): wild type C57BL/6 mice treated with PBS (a healthy mouse model) (WT+PBS, n = 3), wild type mice treated with ADSC-derived EVs (WT+ADSC-EVs, n = 4), APP/PS1 mice (an AD mouse model) treated with PBS (APP/PS1+PBS, n = 4), and APP/PS1 mice treated with ADSC-derived EVs (APP/PS1+ADSC-EVs, n = 4). EVs (5×10⁸ particles/day) or PBS were delivered daily by an IN route continuously for 2 weeks at a dose of 24 µL/mouse (12 µL per nostril) with both nostrils of the mice evenly divided into 4 rounds, and Western blotting was performed to analyzed expression amounts of NLRP3 and caspse-1 after four weeks. Western blotting results showed that in the cortex and hippocampus, expressions of the NLRP3, cleaved caspase-1 (p20) and ASC in the APP/PS1 mouse groups were higher than those in the WT controls. In the WT groups, the ADSC-derived EV group inhibited an expression of NLRP3 by 22.2% (compared to the PBS group); in the APP/PS1 mouse groups, the ADSC-derived EV group inhibited an expression of NLRP3 by 64.3% (compared to the PBS group); in the WT groups, the ADSC-derived EV group inhibited an expression of the cleaved caspase-1 (p20) by 27.0% (compared to the PBS group); and in the APP/PS1 mouse groups, the ADSC-derived EV group inhibited an expression of the cleaved caspase-1 (p20) by 57.5% (compared to the PBS group). From the above results, it can be confirmed that the ADSC-derived EVs may be inhibited by NLRP3 inflammasome, thereby increasing clearance of Aβ, decreasing the formation of Aβ plaques, and treating Alzheimer’s disease.

### Animal Experiments-Intranasal Route (IN Route) Administration Analysis

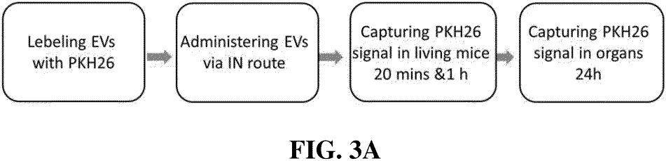

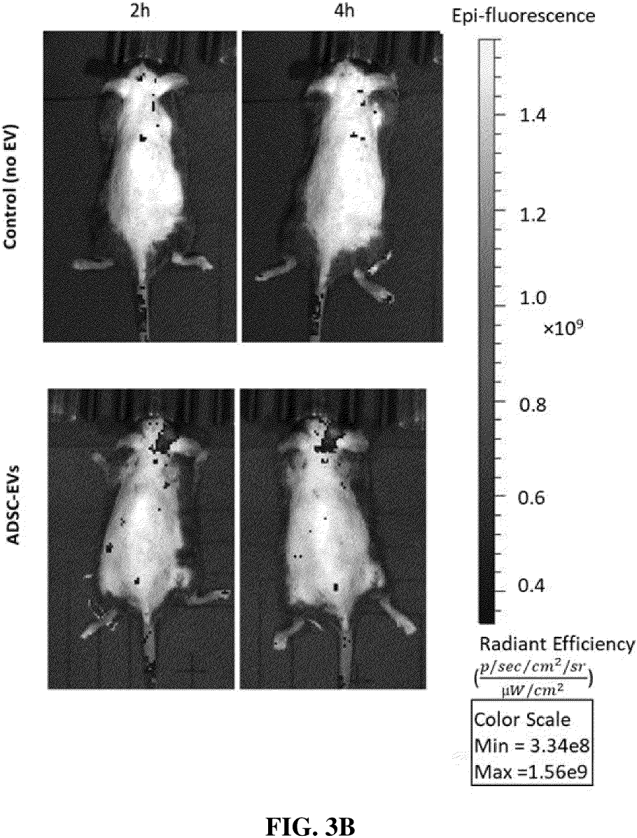

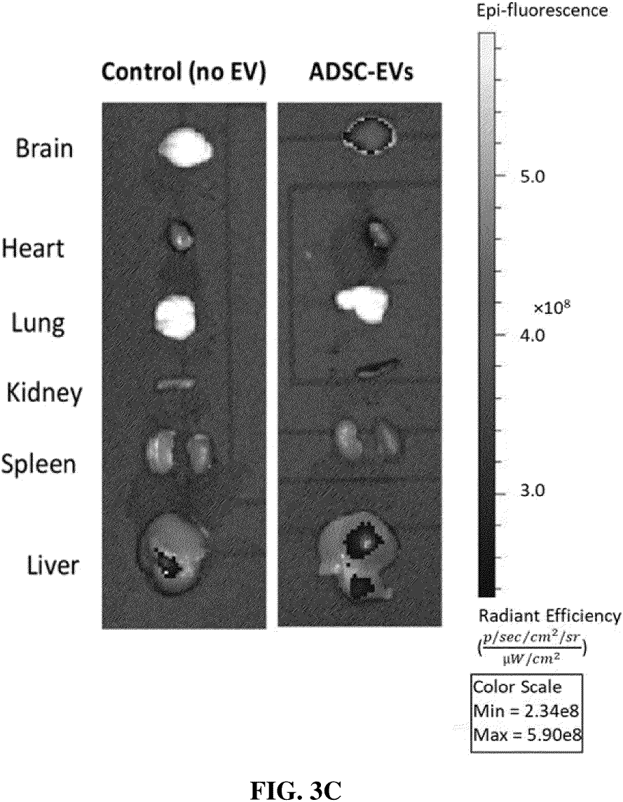

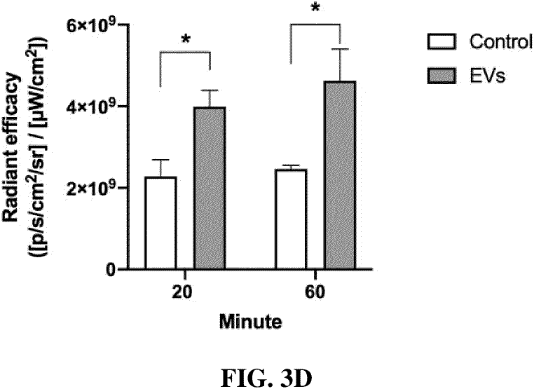

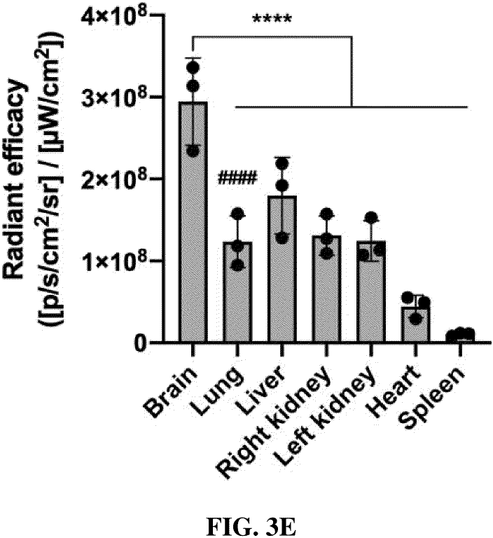

EVs were labeled with PKH26, a fluorescent dye, and then nasally administered at a dose of 2×10¹⁰ particles/mouse (FIG. 3A). Signals were captured and analyzed by an IVIS system. The mice nasally administered with the PKH26-labeled EVs showed an increase in a fluorescence signal between 20 min and 1 hour after the treatment. But the mice nasally administered with PBS showed no significant change in the fluorescence signal (FIGS. 3B-3C). After 24 hours, the experimental mice were euthanized and simply dissected. The brain, heart, lungs, liver, kidneys and spleen were collected to capture distribution of EVs (FIG. 3D). Compared to the lungs (p < 0.0001) and all other organs (p < 0.0001), it was found that the PKH26-labeled EVs were still present and mainly distributed in the brain (FIGS. 3D-3E). This indicates that the intranasal route successfully delivered EVs to a central nervous system.

### Animal Experiments-Side Effect Analysis

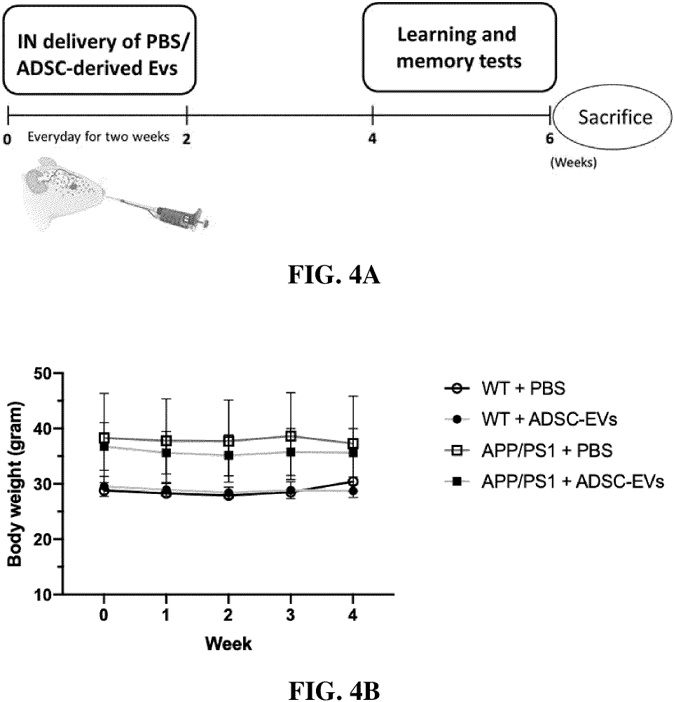

The mice were divided into 4 groups (n = 3-4): wild type C57BL/6 mice treated with PBS (a healthy mouse model) (WT+PBS, n = 3), wild type mice treated with ADSC-derived EVs (WT+ADSC-EVs, n = 4), APP/PS1 mice (an AD mouse model) treated with PBS (APP/PS1+PBS, n = 4), and APP/PS1 mice treated with ADSC-derived EVs (APP/PS1+ADSC-EVs, n = 4). EVs (5×10⁸ particles/day) or PBS were delivered daily by an IN route continuously for 2 weeks at a dose of 24 µL/mouse (12 µL per nostril) with both nostrils of the mice evenly divided into 4 rounds, and a specific operation time course was shown in FIG. 4A.

The mice were monitored weekly for abnormal behavior activities and physical changes throughout a treatment period from week 0 to week 4. Administration positions of each group of the mice had no pathological changes and no inflammatory reactions. In addition, the mice showed significant changes in body weight (p < 0.05) (as shown in FIG. 4B). In summary, our data indicated that EV delivery via the intranasal route did not show adverse side effects on the mice.

### Animal Experiments-Animal Behavior Test Analysis

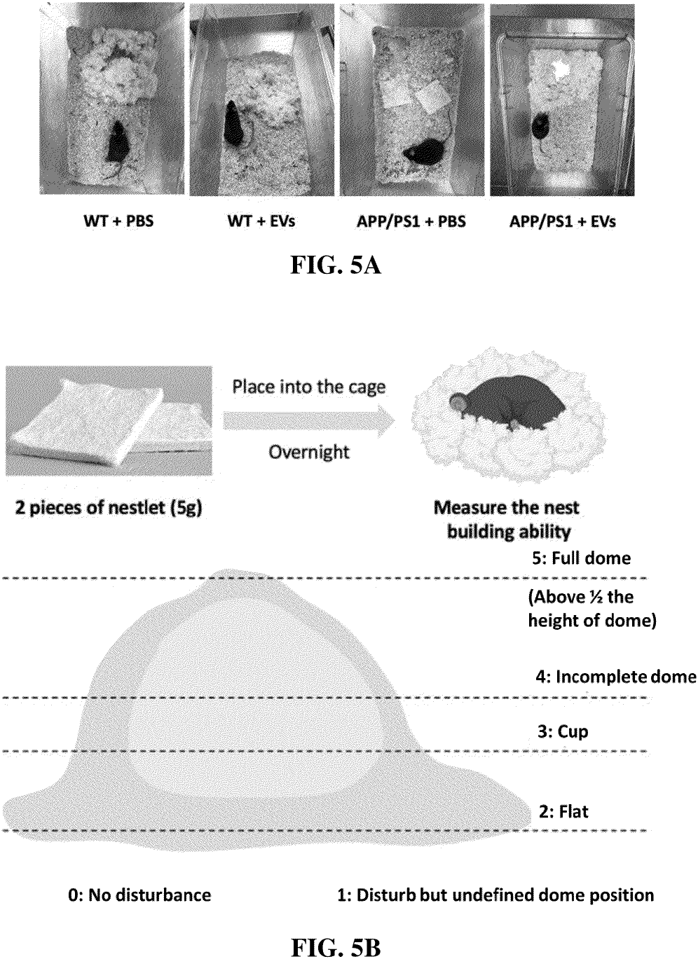

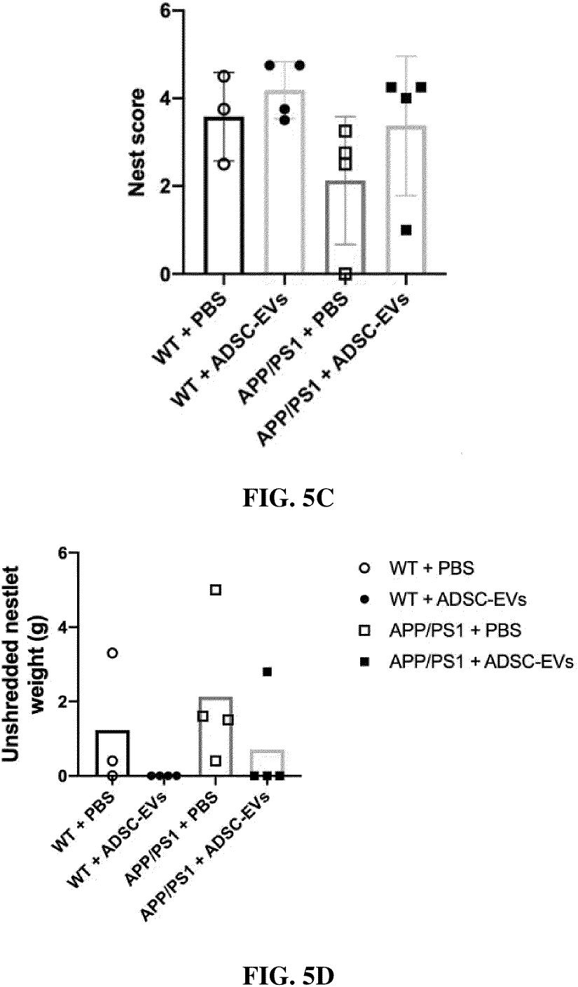

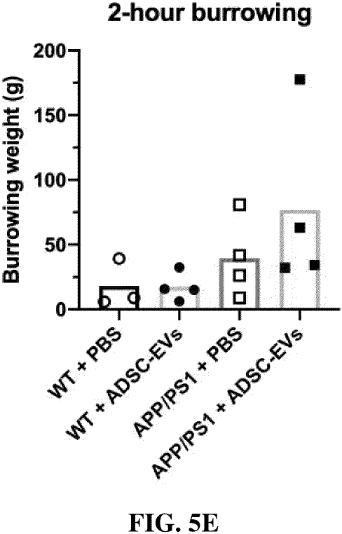

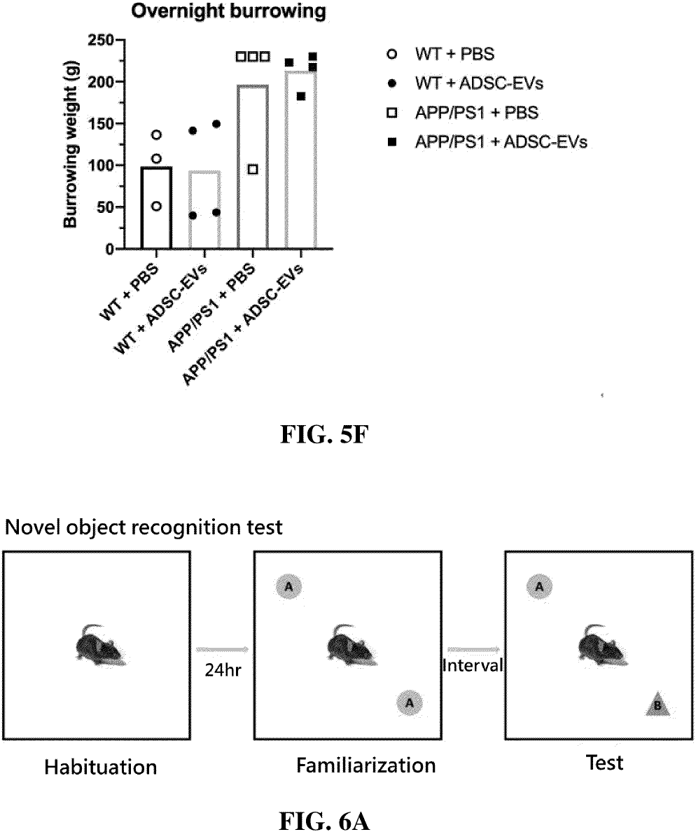

Behavioral disorders associated with cognitive defects are among marks of AD. To assess a performance of activities of daily living (ADL), burrowing and nest building tests were used in an AD mouse model. To perform a nest building test, two pieces of nestlets weighting 5 g were placed in cages 1 hour before a dark cycle. Nests were scored the next day according to a six-score structure form (as shown in FIG. 5B). The nests built by each group were shown in FIG. 5A.

The nest building test method:

One hour before a dark cycle, two pieces of nestlets (5 g) were placed in each mouse cage. A nest building ability was scored based on a six-score structure form: 0 = no disturbance; 1 = disturbance (but undefined nest position in a cage); 2 = flat (difficult to identify a nest wall); 3 = cup nest (a building nest wall is two times shorter than an actual height of a dome); 4 = incomplete dome (a height of a wall is half of that of a dome); and 5 = full dome (a wall of a dome above half the expected height).

The burrowing test method:

2 hours prior to a dark period, each mouse was housed in a single polysulfone cage of 42.5 cm×26.6 cm×18.5 cm in size and a bottom part of the cage was covered with corn cobs. A single end sealed cylinder (made of plastics and a size of 20 cm×7 cm) was placed into the cage and filled with 230 g of regular food particles. After two hours of testing, the food particles remaining in the cylinder were weighed. This step was repeated the next day (after at least 16 hours).

APP/PS1 mice performed poorly in the nest building test with a lower nest score (FIG. 5C) and had a heavier unshredded nestlet weight (FIG. 5D). The ADSC-derived EVs improved the nest building ability but not significantly. Interestingly, the wild type mouse group treated by ADSC-derived EVs did not seem to perform the test better than the wild type mouse group not treated with EVs (FIGS. 5C-5D). In addition, in the burrowing test, at 2 hour (FIG. 5E) and overnight (FIG. 5F) after the start of the test, the APP/PS1 mice treated with ADSC-derived EVs burrowed slightly more food particles than the APP/PS1 mice treated with PBS. However, wild type mice burrowed fewer times than the APP/PS1 mice. It indicated that susceptibility of a cave test varied among mouse strains. In addition, an appropriate group size for the test is ten compared to an experimental scale of each group (three to four mice per group).

### Animal Experiments-Animal Memory Test Analysis

Two weeks after administration, the mice were tested for a cognitive memory by a novel object recognition test.

Novel object recognition test method:

The novel object recognition test comprises 3 stages including a habituation stage, a familiarization stage, and a test stage. The mice were acclimated in a square open area (50 cm×50 cm×50 cm) for 5 min in a dark cycle. After 24 hours of the habituation stage, two identical objects (LEGO building blocks towers) were placed on an open ground 5 cm from a wall. The mice were familiar with the objects in an open field for 10 min. After one-hour interval, an old object was replaced with a new one (a Falcon tissue culture flask filled with Falcon sand) to enable the mice to explore the open field and objects again for 10 min. The entire experiment was recorded by a video camera and the results were used for an analysis.

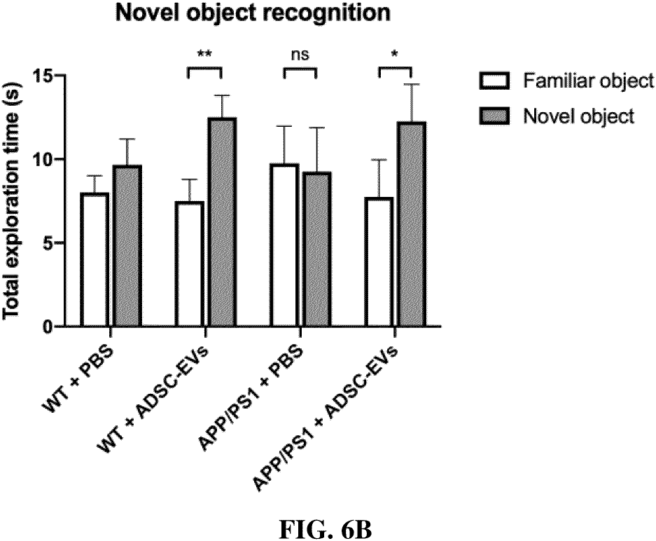

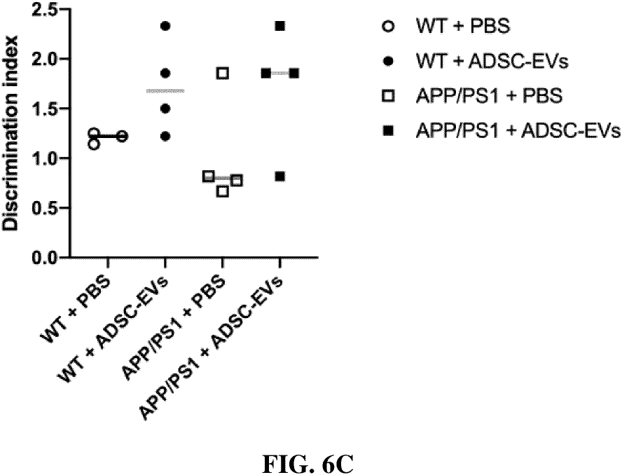

Schematic diagrams of the test were shown in FIG. 6A. Compared with wild type mice, APP/PS1 mice treated with PBS showed a lower cognitive performance in terms of total exploration time of a novel object (FIG. 6B) and a discrimination index of a novel object at the test stage (FIG. 6C). Notably, the exploration time of the novel object was improved (p < 0.05) (FIG. 6B) and the discrimination index was higher (FIG. 6C), but not significant for the APP/PS1 mice treated with ADSC-derived EVs.

These data indicated that ADSC-derived EVs had the potential to rescue a decline of a recognition memory in an AD mouse model.

### Animal Experiments-Aβ Plaque Loading Analysis of APP/PS1 Mice

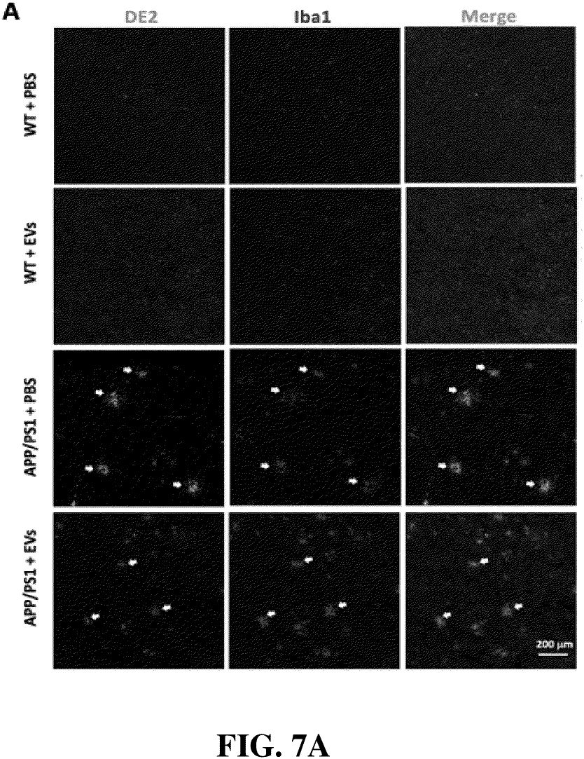

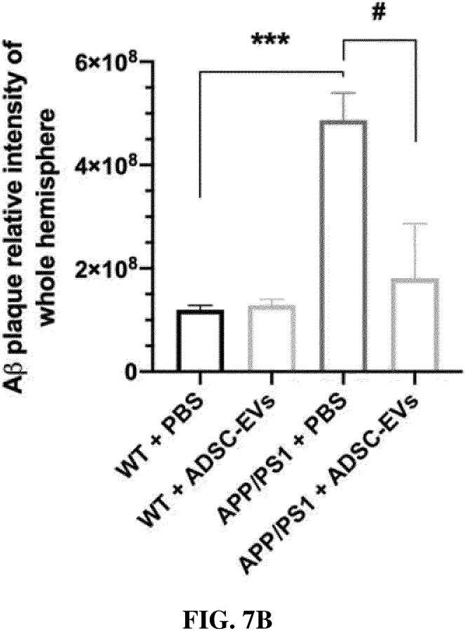

Brains from APP/PS1 mice were collected for immunohistochemistry to assess an expression of Aβ plaques. Aβ plaque loading was detected by immunohistochemical staining with DE2 (anti-amyloid antibody, β1-16, clone DE2, green) and Iba1 (microglia cells, red). The results were shown in FIGS. 7A and 7B (FIG. 7A showed a local enlargement of cortex regions and FIG. 7B showed a quantitative comparison of an Aβ plaque relative intensity of whole hemisphere, data were mean±SEM, the parametric data were analyzed by an unpaired two-tailed Student’s t-test, and significant differences were indicated by * p < 0.05, *** p < 0.001, and # p < 0.05). It can be observed from the figure that Aβ plaques of APP/PS1 mice subjected to IN administration of ADSC-derived EVs were reduced. The APP/PS1 mice had significantly a higher Aβ plaque intensity compared to WT mice. However, compared with the APP/PS1 mice treated with PBS, the EV treatment significantly reduced the Aβ plaque intensity. These results indicated that ADSC-derived EVs had a beneficial effect on relieving Aβ loading in an AD mouse model.

### Animal Experiments-Analysis of Astrocyte Proliferation and Microglia Proliferation of APP/PS1 Mice

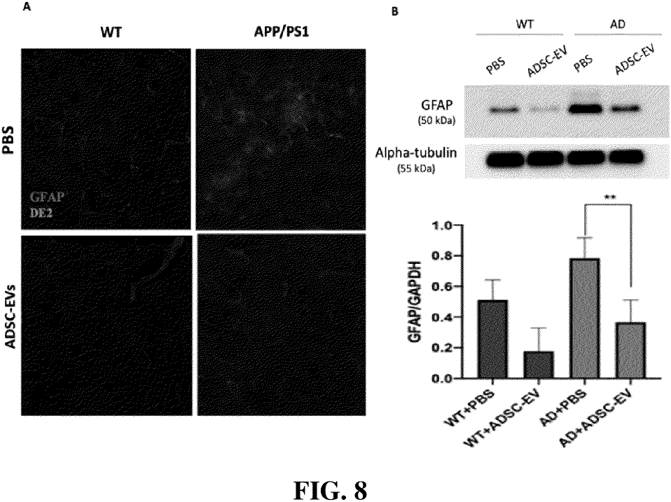

It has been known that activation of astrocytes and microglia is associated with neuroinflammation in the brain of AD. As shown in FIG. 8, an immunohistochemical staining showed that APP/PS1 mice showed a significant increase in an expression of activated astrocytes stained with a GFAP marker (A in the figure showed detection of activated astrocytes, GFAP (blue) and DE2 (anti-amyloid antibody, β1-16, clone DE2, green) in a cortex of WT+PBS, WT+ADSC-EV, APP/PS1+PBS (AD+PBS), and APP/PS1+ADSC-EV (AD+ADSC-EV) by the immunohistochemical staining; B in the figure is an analysis of a cortical lysate by a western blotting analysis of a GFAP expression; and data were mean±SEM, the statistical data compared to the control group were expressed using *, and ** p < 0.01 indicated a significant difference). However, administration of ADSC-derived EVs significantly reduced an expression of GFAP compared treatment of APP/PS1 mice with PBS. Western blotting analysis showed that ADSC-derived EVs reduced a GFAP level increase detected in the APP/PS1 mice. These results indicated that IN administration of the ADSC-derived EVs can reduce neuroinflammation in an AD mouse model.

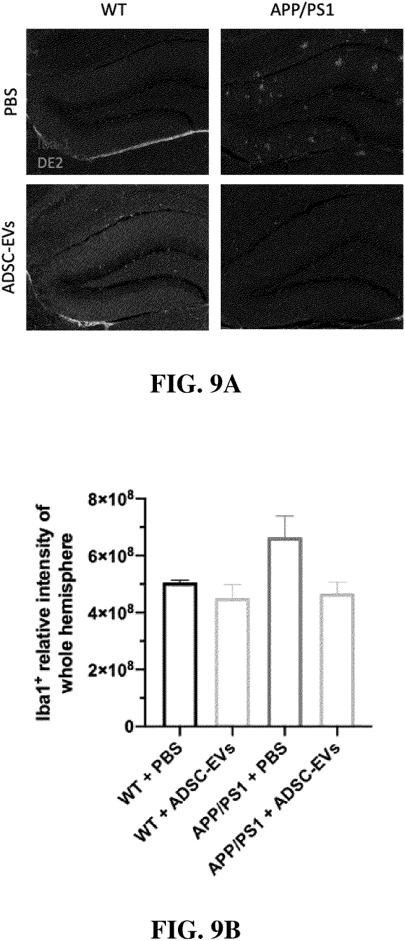

In addition, ADSC-derived EV treatment showed activation of microglia in the APP/PS1 mice was reduced (as shown in FIGS. 9A and 9B, FIG. 9A showed activated microglia in a hippocampus of WT+PBS, WT+ADSC-EV, APP/PS1+PBS (AD+PBS), and APP/PS1+ADSC-EV (AD+ADSC-EV) through detection of Iba-1 (red) and DE2 (Aβ1-16, green) by an immunohistochemical staining, data were mean±SEM, and FIG. 9B shows quantitative data results of FIG. 9A). This was indicated by a reduced expression level of Iba-1. This result further indicated immunosuppressive and anti-inflammatory effects of the ADSC-derived EVs in the brain of AD.

### Animal Experiments-Analysis of Neurogenesis by Transnasal Administration In Vivo

Neurogenesis was measured by an immunohistochemical staining with doublecortin ( projection neurons, red) and BrdU ( neuronal progenitors, green) in a hippocampal portion of mice, including wild type mice treated with PBS (WT+PBS, n = 3), wild type mice treated with ADSC-derived EVs (WT+ADSC-EV, n = 4), APP/PS1 mice treated with PBS (APP/PS1+PBS, n = 4), and APP/PS1 mice treated with ADSC-derived EVs (APP/PS1+ADSC-EV, n = 4) with 1-3 sections per mouse.

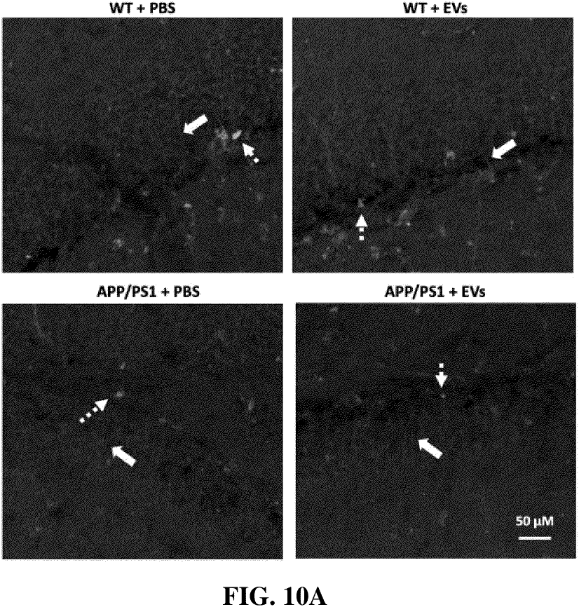

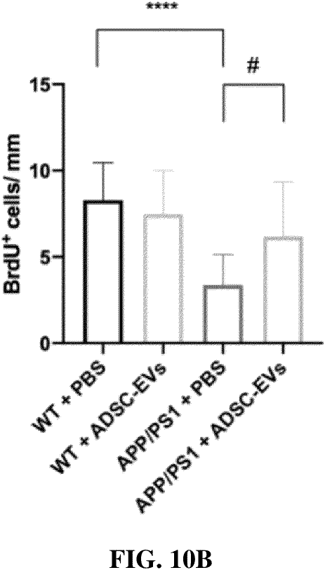

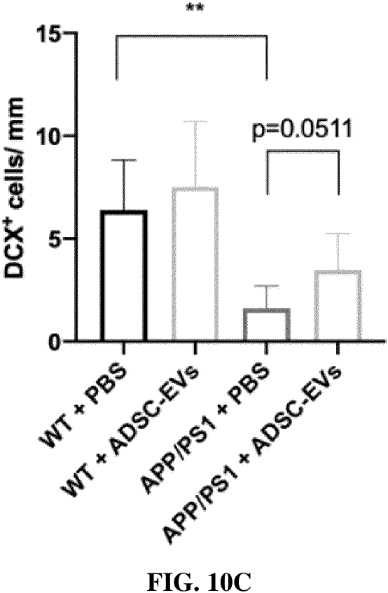

The results were shown in FIGS. 10A to 10C. FIG. 10A showed a partially enlarged view of the SGZ in dentate gyrus, FIG. 10B showed a comparison of the number of BrdU-positive cells, and FIG. 10C showed a comparison of the number of DCX-positive cells. (Data were mean ± SEM. The parametric data were analyzed by an unpaired two-tailed Student’s t-test, and significant differences were indicated by ** p < 0.01, **** p < 0.0001, and # p < 0.05). From the results of FIGS. 10A to 10C, it can be seen that the transnasal administration of ADSC-derived EVs may reduce nerve injury in APP/PS1 mice.

### Example 2

At present, MSC-EV purification produced an average of 500-1,000 particles per cell by ultracentrifugation. In order to further overcome a current problem of EVs production, a nano-channel electroporation (NEP) technology was used. The NEP system consists of an array of nano-channels, each of which has a diameter of 500 nm. A plasmid added to a buffer enters an attached cell through a nano-channel under a transient electrical pulse. The NEP can provide strong but transient electrical and thermal stimulations at a cell surface through a rapid non-endocytic delivery of plasmids, thereby triggering a strong autophagy in a transfected cell, wherein a cell membrane repair directs a massive secretion of endogenously synthesized functional exosomes containing therapeutic RNAs and targeting peptides.

Adipose-derived stem cells at 6,000-15,000 cells/cm² were cultured in a Keratinocyte-SFM medium containing 10 wt% of fetal bovine serum, 1-100 mM of N-acetyl-L-cysteine, and 0.05-50 mM of L-ascorbyl acid 2-phosphate for 5-14 days to expand the number of the cells to 10,000-100,000, wherein the culture was performed at a temperature controlled at 36.5-38.5° C. and in a cell incubator containing 5% carbon dioxide.

Then the cells were rinsed twice with Dulbecco’s phosphate buffered saline (DPBS), 8.0×10⁴ of the adipose-derived stem cells were spread on a surface of a 3D NEP silicon chip of 1 cm×1 cm, wherein a serum-free culture medium was a Keratinocyte-SFM culture medium containing 1-100 mM of N-acetyl-L-cysteine and 0.05-50 mM of L-ascorbyl acid 2-phosphate, after the cells adhered to walls, an electroporation system (Bio-Rad Gene Pulser Xcell) under a condition of a square-wave pulsed electric field of 125 V and a duration of 10 ms for 10 times of pulses was used, a culture was performed at a temperature controlled at 36.5-38.5° C. and in a cell incubator containing 5% carbon dioxide, and after 24 hours, the culture medium was collected for an extracellular vesicle purification.

A culture solution after the completion of the culture was collected and centrifuged at a centrifugation speed of 300 g at a temperature of 4° C. for 5 min to remove dead cells in the culture solution; and then after a supernatant was collected, the supernatant was centrifuged at a centrifugation speed of 2,000 g at a temperature of 4° C. for 20 min to remove cell debris, then the supernatant was collected and filtered through a filter screen having a pore size of 0.22 µm, extracellular vesicles were separated from the filtered culture solution by tangential flow filtration (TFF, 500 Ka)and recorded in Table 3.

The results were shown in Table 3. There was no significant difference between different donors and different passage numbers under a serum-free condition (as shown in Table 3). The yield of secreted extracellular vesicles (EVs) was increased 2-5 times compared to a conventional culture condition.

TABLE 3

| Groups | Sources of EVs | Passage number of cells | Concentration of EVs (particle/cell) |
|---|---|---|---|
| 17 | ADSCs#1 | P12 | 1,858 |
| 18 | ADSCs#1 | P13 | 2,195 |
| 19 | ADSCs#2 | P10 | 1,268 |
| 20 | ADSCs#2 | P11 | 2,138 |
| 21 | ADSCs#3 | P9 | 1,054 |
| 22 | ADSCs#3 | P10 | 886 |
| 23 | ADSCs#3 | P14 | 1,980 |

### Example 3

The adipose-derived stem cells at 6,000-15,000 cells/cm² were cultured in a Keratinocyte-SFM medium containing 10 wt% of fetal bovine serum, 1-100 mM of N-acetyl-L-cysteine, and 0.05-50 mM of L-ascorbyl acid 2-phosphate for 5-14 days to expand the number of the cells to 10,000-100,000, wherein the culture was performed at a temperature controlled at 36.5-38.5° C. and in a cell incubator containing 5% carbon dioxide.

Then the cells were rinsed twice with Dulbecco’s phosphate buffered saline (DPBS), 8.0×10⁴ of the adipose-derived stem cells were spread on a surface of a 3D NEP silicon chip of 1 cm×1 cm, wherein a serum-free culture medium was a Keratinocyte-SFM culture medium containing 1-100 mM of N-acetyl-L-cysteine and 0.05-50 mM of L-ascorbyl acid 2-phosphate, after the cells adhered to walls, an electroporation system (Bio-Rad Gene Pulser Xcell) under a condition of a square-wave pulsed electric field of 125 V and a duration of 10 ms for 10 times of pulses was used, a neprilysin expression plasmid (product name: CD10 (MME) (NM_000902) human tagged ORF clone purchased from OriGene Technologies, Inc.) added in a PBS buffer solution in advance was injected into a single cell through a nano-channel, a culture was performed at a temperature controlled at 36.5-38.5° C. and in a cell incubator containing 5% carbon dioxide, and after 24 hours, the culture medium was collected for an extracellular vesicle purification. An mRNA of a neprilysin in the PBS had a concentration of 500 ng/ml.

A culture solution after the completion of the culture was collected and centrifuged at a centrifugation speed of 300 g at a temperature of 4° C. for 5 min to remove dead cells in the culture solution; and then after a supernatant was collected, the supernatant was centrifuged at a centrifugation speed of 2,000 g at a temperature of 4° C. for 20 min to remove cell debris, then the supernatant was collected and filtered through a filter screen having a pore size of 0.22 µm, extracellular vesicles were separated from the filtered culture solution by tangential flow filtration (TFF, 500 Ka), and an expression amount of a neprilysin (analyzed by Western blotting and quantified by an Image J software) in extracellular vesicles was analyzed and recorded in Table 4.

TABLE 4

| Groups | Concentration of extracellular vesicles | Expression amount of neprilysin | Expression amount of neprilysin/control group (%) |
|---|---|---|---|
| Control | NA | 0.062±0.0017 | 100 |
| ADSCs naive | 1×10⁷ | 116.03±32.82 | 1,866±528 |
| ADSC NEP-PBS | 1×10⁷ | 122.30±34.59 | 1,966±556 |
| ADSC NEP-MMA | 1×10⁷ | 185.64±52.51 | 2,985±884 |

The samples of each group in Table 4 were described as follows:

Control group: treated with PBS.

ADSCs naive group: extracellular vesicles derived from adipose-derived stem cells using the method of example 1.

ADSC NEP-PBS group: extracellular vesicles obtained using the method of this example without adding any plasmids in PBS.

ADSC NEP-MME group: extracellular vesicles obtained by the method of this example and adding an mRNA of a neprilysin in PBS.

As can be seen from the results in Table 4, there was almost no neprilysin in the control group, the expression amount of the neprilysin was the same in the ADSCs naive group and the ADSC NEP-PBS group, and the expression amount of the neprilysin in the ADSC NEP-MME group was about 1.5-1.6 times the that in the ADSCs naive group or the ADSC NEP-PBS group.

Subsequently, effects of extracellular vesicles on an APP gene mutant induced pluripotent stem cell (APP^{V1711} mutant AD-iPSCs or APP^{D678H} mutant AD-iPSCs) and a PSEN1 gene mutant induced pluripotent stem cell (PSEN1^{A246E} mutant AD-iPSCs) respectively were confirmed. Both the APP gene and the PSEN1 gene are associated with Alzheimer’s disease. Hereditary Alzheimer’s disease is caused by a group of genetic variations on chromosomes 21, 14 and 1 and abnormal proteins formed by these variations. An APP gene mutation on chromosome 21 results in the formation of an abnormal amyloid precursor protein and a PSEN1 gene mutation on chromosome 14 results in abnormal presenilin 1.

APP^{V1711} mutant AD-iPSCs and PSEN1^{A246E} mutant AD-iPSCs (related materials and methods such as “Assessing the therapeutic potential of Graptopetalum paraguayense on Alzheimer’s disease using patient iPSC-derived neurons”. Sci Rep. 2019 Dec 17; 9(1): 19301) were treated with different extracellular vesicles, including the extracellular vesicles derived from adipose-derived stem cells using the method of example 1 (ADSCs naive group), extracellular vesicles obtained using the method of this example without adding any plasmids in PBS (ADSC NEP-PBS group), extracellular vesicles obtained by the method of this example and adding an mRNA of a neprilysin in PBS (ADSC NEP-MMA group), wherein the extracellular vesicles of all groups had a concentration of 1.3×10⁹ or 6×10⁹ particles. After two days of culture, the culture medium was replaced with a fresh culture medium and extracellular vesicles of the same concentration as the previous were added and then cultured for two days. Finally, the culture medium was collected and an expression degree of Aβ1-42 was analyzed (by an ELISA assay).

APP-D678H mutant AD-iPSCs were differentiated into neurons, the culture medium after 12 weeks of differentiation (hereinafter referred as a 12 W culture medium) was collected, and the 12 W culture medium or an Aβ standard protein (amyloid-β standard protein) was mixed with 2×10⁹ or 1×10¹⁰ extracellular vesicles for culturing for 16 hours, wherein the culture was performed at a temperature controlled at 36.5-38.5° C. and in a cell incubator containing 5% carbon dioxide. Finally, the culture medium was collected and an expression degree of Aβ1-42 was analyzed (by an ELISA assay).

The results showed that the ADSC naive, ADSC-NEP-PBS or ADSC-NEP-MME in the three different AD-iPSCs had a similar effect on Aβ1-42, and can all effectively inhibit the expression of the Aβ1-42. It can be seen from this that the extracellular vesicles derived from adipose-derived stem cells using the method of example 1 (ADSC naive and ADSC-NEP-PBS) can express the neprilysin and can inhibit the expression of the Aβ1-42 equivalently compared with those after transfection (ADSC-NEP-MME). Therefore, the present disclosure did not need to additionally transfect the neprilysin, and the extracellular vesicles with a high expression of the neprilysin can be obtained.

### Example 4

The adipose-derived stem cells at 6,000-15,000 cells/cm² were cultured in a Keratinocyte-SFM medium containing 10 wt% of fetal bovine serum, 1-100 mM of N-acetyl-L-cysteine, and 0.05-50 mM of L-ascorbyl acid 2-phosphate for 5-14 days to expand the number of the cells to 10,000-100,000, wherein the culture was performed at a temperature controlled at 36.5-38.5° C. and in a cell incubator containing 5% carbon dioxide.

Then the cells were rinsed twice with Dulbecco’s phosphate buffered saline (DPBS), 8.0×10⁴ of the adipose-derived stem cells were spread on a surface of a 3D NEP silicon chip of 1 cm×1 cm, wherein a serum-free culture medium was a Keratinocyte-SFM culture medium containing 1-100 mM of N-acetyl-L-cysteine and 0.05-50 mM of L-ascorbyl acid 2-phosphate, after the cells adhered to walls, an electroporation system (Bio-Rad Gene Pulser Xcell) under a condition of a square-wave pulsed electric field of 125 V and a duration of 10 ms for 10 times of pulses was used, an miR29b expression plasmid (product name: miR29B-2 human microRNA expression plasmid (MI0000107) purchased from OriGene Technologies, Inc.) added in a PBS buffer solution in advance was injected into a single cell through a nano-channel, a culture was performed at a temperature controlled at 36.5-38.5° C. and in a cell incubator containing 5% carbon dioxide, and after 24 hours, the culture medium was collected for an extracellular vesicle purification. miR29b in the PBS had a concentration of 500 ng/ml.

A culture solution after the completion of the culture was collected and centrifuged at a centrifugation speed of 300 g at a temperature of 4° C. for 5 min to remove dead cells in the culture solution; and then after a supernatant was collected, the supernatant was centrifuged at a centrifugation speed of 2,000 g at a temperature of 4° C. for 20 min to remove cell debris, then the supernatant was collected and filtered through a filter screen having a pore size of 0.22 µm, extracellular vesicles were separated from the filtered culture solution by tangential flow filtration (TFF, 500 Ka), and an expression amount of miR29b (analyzed by a RT-PCR) in extracellular vesicles was analyzed. As a result, it was found that the miR29b was hardly expressed in a control group and the expression amount of the miR29b in an ADSC NEP-miR29b group was 5,496±3,530 times that of the control group.

APP^{V1711} mutant AD-iPSCs and PSEN1^{A246E} mutant AD-iPSCs were treated with the extracellular vesicles (1.3×10⁹ or 6×10⁹ particles) obtained using the method of this example and adding miR29b in PBS. After two days of culture, the culture medium was replaced with a fresh culture medium, and extracellular vesicles of the same concentration as the previous were added and then cultured for two days. Finally, the culture medium was collected and an expression degree of Aβ1-42 was analyzed (by an ELISA assay).

As a result, it was found that an expression of Aβ1-42 (2.58 pmol/L) in an APP^{V1711} mutant AD-iPSCs group treated with 1.3×10⁹ of ADSC NEP-miR29-EVs was reduced by 73.8% compared with an untreated group (9.83 pmol/L); and the expression of Aβ1-42 (4.49 pmol/L) in the APP^{V1711} mutant AD-iPSCs group treated with 6×10⁹ of ADSC NEP-miR29-EVs was reduced by 48.1% compared with the untreated group (8.65 pmol/L). The expression of Aβ1-42 (1.69 pmol/L) in an PSEN1^{A246E} mutant AD-iPSCs group treated with 1.3×10⁹ of ADSC NEP-miR29-EVs was reduced by 58.9% compared with an untreated group (4.11 pmol/L); and the expression of Aβ1-42 (1.3 pmol/L) in the PSEN1^{A246E} mutant AD-iPSCs group treated with 6×10⁹ of ADSC NEP-miR29-EVs was reduced by 68.4% compared with the untreated group (4.11 pmol/L).

APP-D678H mutant AD-iPSCs were differentiated into neurons, the culture medium after 12 weeks of differentiation (hereinafter referred as a 12 W culture medium) was collected, and the 12 W culture medium or an Aβ standard protein (amyloid-β standard protein) was mixed with 2×10⁹ or 1×10¹⁰ extracellular vesicles (ADSC NEP-miR29-EVs) for culturing for 16 hours, wherein the culture was performed at a temperature controlled at 36.5-38.5° C. and in a cell incubator containing 5% carbon dioxide. Finally, the culture medium was collected and an expression degree of Aβ1-42 was analyzed (by an ELISA assay).

As a result, it was found that the expression of Aβ1-42 (40.3 pg/ml) of a 12 W culture medium and 2×10⁹ ADSC NEP-miR29-EVs group was reduced by 91.9% compared with an untreated group (496 pg/ml); and the expression of Aβ1-42 (8 pg/ml) of a 12 W culture medium and 1×10¹⁰ ADSC NEP-miR29-EVs group was reduced by 98.4% compared with the untreated group (496 pg/ml). The expression of Aβ1-42 (638 pg/ml) of an Aβ standard protein and 2×10⁹ ADSC NEP-miR29-EVs group was reduced by 35.3% compared with an untreated group (987 pg/ml); and the expression of Aβ1-42 (257 pg/ml) of an Aβ standard protein and 1×10¹⁰ ADSC NEP-miR29-EVs group was reduced by 73.9% compared with the untreated group (987 pg/ml). As described in the above results, the extracellular vesicles with miR29 can effectively inhibit the expression of Aβ1-42.

In conclusion, the content of the present disclosure has been exemplified with reference to the above examples, but the present disclosure is not limited to the embodiments. A person with common knowledge in the art may make various changes and modifications without departing from the spirit and scope of the present disclosure, for example, the technical contents exemplified in the above examples are combined or changed to new embodiments, and these embodiments are also regarded as one of the contents of the present disclosure. Accordingly, the scope to be protected in this case also includes the scope of aftermentioned claims and the scope defined therein.

### REFERENCE NUMERALS

None

1. A pharmaceutical composition for treating Alzheimer’s disease, which at least comprises an extracellular vesicle that is prepared by a method comprising:
a first culturing step: performing an amplification culture of an adipose-derived stem cell in a first culture medium at a cell density of 6,000-15,000 cells/cm² until an amplification amount of the adipose-derived stem cell is above 90% of that before the culture;
a second culturing step: culturing the amplified adipose-derived stem cell in a second culture medium at a cell density of 10,000-100,000 cells/cm² for 20-30 hours; and
an extracellular vesicle separating step: collecting a culture solution and separating the extracellular vesicle from the culture solution by utilizing a tangential flow filtration (TFF) or ultrafiltration method, wherein
the first culture medium is a culture medium containing fetal bovine serum, N-acetyl-L-cysteine, L-ascorbic acid 2-phosphate, and Keratinocyte-SFM; and
the second culture medium is a culture medium containing N-acetyl-L-cysteine, L-ascorbic acid 2-phosphate, and Keratinocyte-SFM culture medium.

2. The pharmaceutical composition for treating Alzheimer’s disease according to claim 1, wherein after the second culturing step, the method further comprises:
a gene transfection step: spreading the adipose-derived stem cell on a surface of a 3D NEP silicon chip, after the cell is adhered, treating the cell by using an electroporation system under a condition of a square-wave pulsed electric field of 25-150 V and a duration of 10 ms for 5-15 times of pulses, injecting a plasmid added in a PBS buffer solution in advance into a single cell through a nano-channel, and culturing the cell for 20-30 hours to perform the extracellular vesicle separating step.

3. The pharmaceutical composition for treating Alzheimer’s disease according to claim 2, wherein the plasmid in the PBS has a concentration between 400-600 ng/ml.

4. The pharmaceutical composition for treating Alzheimer’s disease according to claim 1, wherein the extracellular vesicle highly expresses neprilysin.

5. The pharmaceutical composition for treating Alzheimer’s disease according to claim 2, wherein the extracellular vesicle highly expresses neprilysin, miR-29a and/or miR-29b.

6. The pharmaceutical composition for treating Alzheimer’s disease according to claim 1, wherein the pharmaceutical composition is administered transnasally.

7. A method for culturing a high-phenotype mesenchymal stem cell, which comprises the following steps:
a first culturing step: performing an amplification culture of an adipose-derived stem cell in a first culture medium at a cell density of 6,000-15,000 cells/cm² until an amplification amount of the adipose-derived stem cell is 90% or more of that before the culture;
a second culturing step: culturing the amplified adipose-derived stem cell in a second culture medium at a cell density of 10,000-100,000 cells/cm² for 20-30 hours; and
a gene transfection step: spreading the adipose-derived stem cell on a surface of a 3D NEP silicon chip, after the cell is adhered, treating the cell by using an electroporation system under a condition of a square-wave pulsed electric field of 120-150 V and a duration of 10 ms for 5-15 times of pulses, injecting a plasmid added in a PBS buffer solution in advance into a single cell through a nano-channel, and culturing the cell for 20-30 hours to obtain a high-phenotype mesenchymal stem cell, wherein
the plasmid is an mRNA of neprilysin, miR-29a and/or miR-29b; and
the high-phenotype mesenchymal stem cell highly expresses neprilysin, miR-29a and/or miR-29b.

8. The method for culturing a high-phenotype mesenchymal stem cell according to claim 7, wherein the plasmid in the PBS has a concentration between 400-600 ng/ml.

9. The method for culturing a high-phenotype mesenchymal stem cell according to claim 7, wherein the high-phenotype mesenchymal stem cell can be used for treating Alzheimer’s disease and/or amyloidosis.

10. The method for culturing a high-phenotype mesenchymal stem cell according to claim 7, wherein the extracellular vesicle derived from the high-phenotype mesenchymal stem cell can be used for treating Alzheimer’s disease and/or amyloidosis.
